(19) United States
(12) Patent Application Publication
(10) Pub. No.: US 2022/0034244 A1
(43) Pub. Date: Feb. 3, 2022

(54) SOUND SUPPRESSION DEVICE FOR GAS CAPTURE SYSTEM OF CYCLOTRON PRODUCT

(71) Applicant: Dana-Farber Cancer Institute, Inc., Boston, MA (US)

(72) Inventor: Erol Bars, Exeter, NH (US)

(21) Appl. No.: 17/297,829

(22) PCT Filed: Nov. 27, 2019
(86) PCT No.: PCT/US19/63585

§ 371 Date: May 27, 2021

Related U.S. Application Data
(60) Provisional application No. 62/773,727, filed on Nov. 30, 2018.

(51) Int. Cl. F01N 1/10; G21F 9/02; H05H 13/00

(52) U.S. Cl. CPC F01N 1/10; F01N 2310/14; H05H 13/005; G21F 9/02

(57) ABSTRACT

A cyclotron sound suppression device for reducing the decibel level of the supersonic exhaust gases. A cyclotron unit for preparing a radioisotope includes a storage tank for storing a radioactive gas resulting from preparation of the radioisotope, a compressor connected with the storage tank(s); an exhaust valve in connected with the storage tank(s); and a sound suppression device in connected with the exhaust valve. The sound suppression device can be configured as a supersonic muffler and attached to the outlet valve to diffuse the exhaust gases, thereby reducing risk of hearing damage.

### CROSS-REFERENCE TO RELATED SUBJECT MATTER

This application claims the benefit under 35 USC 119 of U.S. Provisional Application No. 62/773,727, filed Nov. 30, 2018, which is hereby incorporated by reference in its entirety.

### BACKGROUND OF THE DISCLOSED SUBJECT MATTER

### Field of the Disclosed Subject Matter

The disclosed subject matter relates to a system for sound suppression of exhaust gases in a cyclotron. Particularly, the present disclosed subject matter is directed toward a muffler capable of diffusing super sonic exhaust gas streams.

### DESCRIPTION OF RELATED ART

The present disclosure is directed towards the field of Positron Emission Tomography (PET), which includes imaging and measuring physiologic processes by injecting radioisotopes into a patient to assist in diagnosing and assessing disease progression/treatment. A cyclotron or particle accelerator is used to produce the radioisotopes. Conventional cyclotrons accelerate the particle beam and thereafter collide or bombard a target material (e.g. solid, liquid or gaseous) which is housed in a target holder or container of the cyclotron. The generation of the radioisotope creates fluid waste containing radioisotopes, which requires a device for safely collecting and containing the waste until the radioisotopes have sufficiently decayed to the extent that they can be safely released into the atmosphere.

These short-lived radioisotopes are either directly released to the general atmosphere or are first stored in highly pressurized containers before being released—with the pressure differential between storage tank and atmosphere causing gases to exit at excessive speeds and harmful decibel levels.

There thus remains a need for an efficient and economic method and system for venting the stored gas to atmosphere while minimizing the noise pollution.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a cyclotron system comprising: a cyclotron unit for preparing a radioisotope; at least one storage tank for storing a radioactive gas resulting from preparation of the radioisotope; a compressor connected with the storage tank(s); an exhaust valve connected with the storage tank(s); and a sound suppression device connected with the exhaust valve.

In some embodiments, the compressor compresses the internal pressure of the storage tank above approximately 10 bars.

In some embodiments, the sound suppression device has a primary internal channel with a diameter approximately equivalent to the diameter of the exhaust valve.

In some embodiments, the primary internal channel is configured as a linear channel aligned with the longitudinal axis of the sound suppression device.

In some embodiments, the sound suppression device includes a plurality of diffusion channels distributed along the length of the device and connected with the primary internal channel.

In some embodiments, the diffusion channels extend radially from the primary channel.

In some embodiments, diffusion channels are uniformly distributed along the longitudinal axis of the sound suppression device.

In some embodiments, exhaust gas is vented through the sound suppression device traveling through the primary and secondary channels.

In some embodiments, the sound suppression device generates an exhaust gas of approximately 65-75 decibels.

In some embodiments, the sound suppression device is made of nylon.

In accordance with another aspect of the disclosure, a sound suppression device is provided and configured for coupling to an exhaust valve of a cyclotron comprising: a first flange, the first flange having a plurality of apertures disposed therein; a second flange, the second flange having a plurality of apertures disposed therein; a generally cylindrical length defined between the first and second flanges; wherein a primary internal channel extends throughout the cylindrical length of the device.

In some embodiments, the diameter of the primary internal channel is approximately equivalent to the diameter of an exhaust valve of the cyclotron.

In some embodiments, the primary internal channel is configured as a linear channel aligned with a longitudinal axis of the device.

In some embodiments, the device includes a plurality of diffusion channels distributed along the length of the device each c connected with the primary internal channel.

In some embodiments, the diffusion channels extend radially from the primary channel. In some embodiments, diffusion channels are uniformly distributed along the longitudinal axis of the device. In some embodiments, diffusion channels have uniform diameters. In some embodiments, diffusion channels include a first set of channels having a first diameter, and a second set of channels having a second diameter. In some embodiments, diffusion channels are linear channels.

In some embodiments, second flange has a greater number of apertures than the first flange.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1-2 are schematic representations of an exemplary cyclotron systems which can be employed in connection with the radioisotope production system disclosed herein.

FIG. 3 is a schematic representation of an exemplary cyclotron apparatus including moveable doors, shown in an open configuration, in accordance with the disclosed subject matter.

FIG. 4 is a photographic and schematic illustration of a cyclotron exhaust system which separates the portion of the exhaust containing radiation.

FIG. 5 is a schematic representation of the cyclotron exhaust system for the portion of the exhaust containing radiation.

FIG. 6 is a photographic illustration of the cyclotron exhaust system with a sound suppression device in accordance with the disclosed subject matter.

FIG. 7 is a schematic representation of the sound suppression device in accordance with the disclosed subject matter.

FIGS. 8-12 are various cross-sectional views of the exemplary sound suppression device of FIG. 5.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

The present disclosure is directed towards a radioisotope production system that receives the output from a cyclotron, which is a type of particle accelerator in which a beam of charged particles (e.g., H-charged particles or D-charged particles) are accelerated outwardly along a spiral orbit. The cyclotron directs the beam into a target material to generate the radioisotopes (or radionuclides). Cyclotrons are known in the art, and an exemplary cyclotron is disclosed in U.S. Pat. No. 10,123,406, the entirety, including structural components and operational controls, is hereby incorporated by reference.

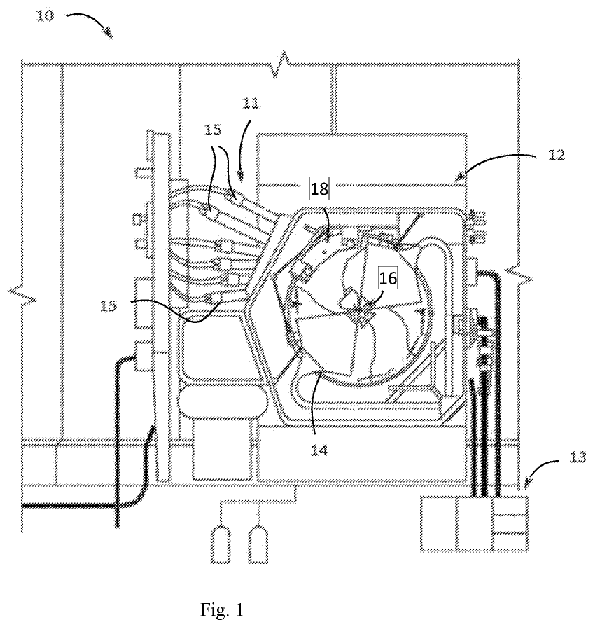

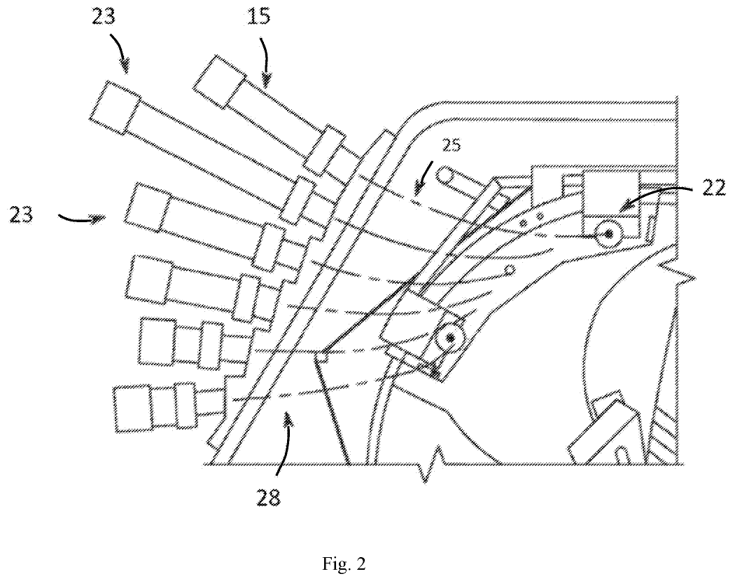

For example, FIG. 1 depicts an exemplary cyclotron construction in which the particle beam is directed by the radioisotope production system 10 through the extraction system 18 along a beam transport path and into the target system 11 so that the particle beam is incident upon the designated target material (solid, liquid or gas). In this exemplary configuration, the target system 11 includes six potential target locations 15, however a greater/lesser number of target locations 15 can be employed as desired. Similarly, the relative angle of each target location 15 relative to the cyclotron body can be varied (e.g. each target location 15 can be angled over a range of 0°˜90° with respect to a horizontal axis in FIG. 2). Additionally, the radioisotope production system 10 and the extraction system 18 can be configured to direct the particle beam along different paths toward the target locations 15.

FIG. 2 is a zoom-in side view of the extraction system 18 and the target system 11. In the illustrated embodiment, the extraction system 18 includes first and second extraction units 22. The extraction process can include stripping the electrons of the charged particles (e.g., the accelerated negative charged particles) as the charged particles pass through an extraction foil—where the charge of the particles is changed from a negative charge to a positive charge thereby changing the trajectory of the particles in the magnet field. Extraction foils may be positioned to control a trajectory of an external particle beam 25 that includes the positively-charged particles and may be used to steer the external particle beam 25 toward designated target locations 15. These target locations can include solid, liquid or gas targets.

In general, cyclotrons accelerate charged particles (e.g., hydrogen ions) using a high-frequency alternating voltage. A perpendicular magnetic field causes the charged particles to spiral in a circular path such that the charged particles re-encounter the accelerating voltage many times. The magnetic field maintains these ions in a circular trajectory and a D-shaped electrode assembly creates a varying RF electric field to accelerate the particles. As noted above, the cyclotron further includes a beam extraction system consists of a stripper foil, which changes the ion polarity to positive and directs the positively charged ions to hit a target material contained in a target container according to a target selection setting.

The methods and systems presented herein may be used for venting gases from cyclotron storage tanks. For purpose of explanation and illustration, and not limitation, an exemplary embodiment of the system in accordance with the disclosed subject matter is shown in the attached drawings. Similar reference numerals (differentiated by the leading numeral) may be provided among the various views and Figures presented herein to denote functionally corresponding, but not necessarily identical structures.

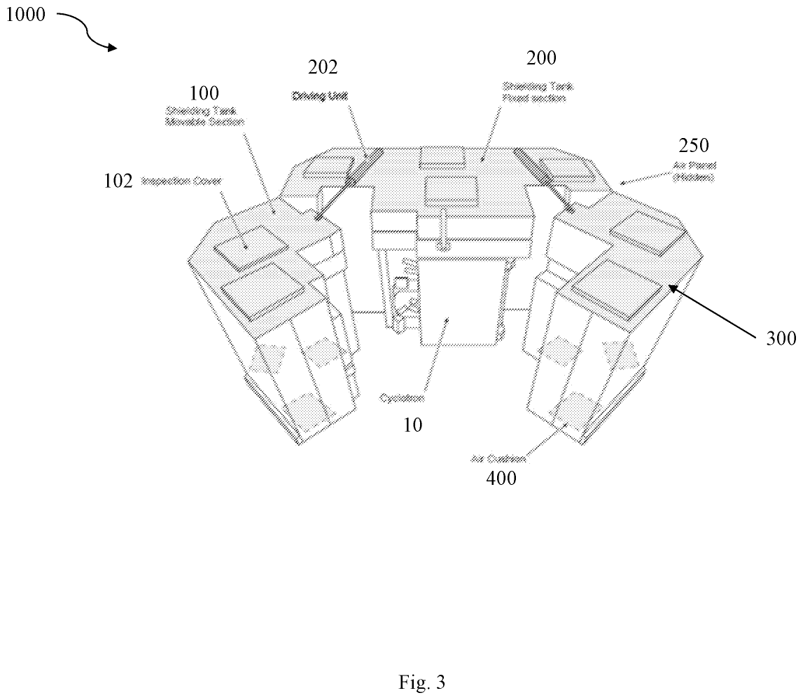

As shown in FIG. 3, the system 1000 depicts a general configuration for shielding a cyclotron 10, with the cyclotron 10 positioned between movable shields 100 and 300 which operate like doors, via driving unit to open to expose the cyclotron 10, and close to contain the cyclotron within the “housing” and serve as shields to the radiation generated therein. The moveable shield doors 100, 300 are hingedly attached to the fixed base shielding section 200. A driving unit 202 can be provided on the top surface of the housing and operated via hydraulics, pneumatics, or electric motor to extend a telescoping piston in order to pivot the doors 100, 300 to rotate open and closed. The doors 100, 300 as well as the base 200 can be configured as semi-hollow tanks which are filled with a medium (e.g. water mixed with boron and lead) to increase the density of the structure and thereby enhance the shielding effect. To offset this increased weight from the filled tanks, inflatable (e.g. air) cushions 400 can be provided on the bottom surfaces of the moveable doors 100, 300 to reduce friction and facilitate gliding of the tanks during the opening/closing movement.

In operation, the cyclotron 10 generates a particle beam that bombards target material located within target enclosure housed within the cyclotron 10 to produce a radioactive isotope which then decays. The decay of the isotope as well as other interactions generates gamma and neutron radiation that is reduced by the shields 100, 200, 300 to protect personnel in the vicinity of the cyclotron against unsafe levels of radiation.

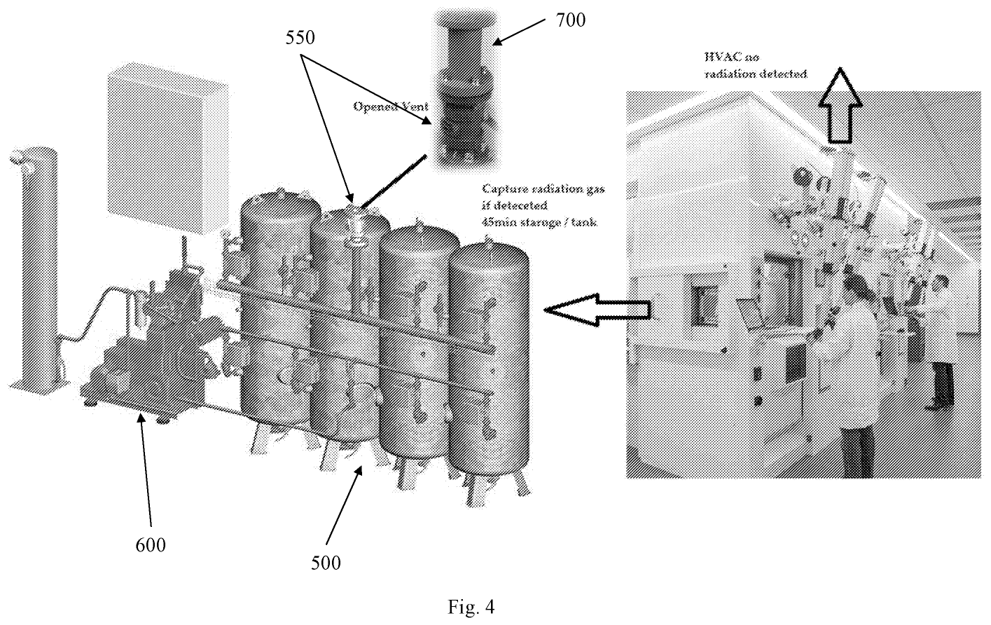

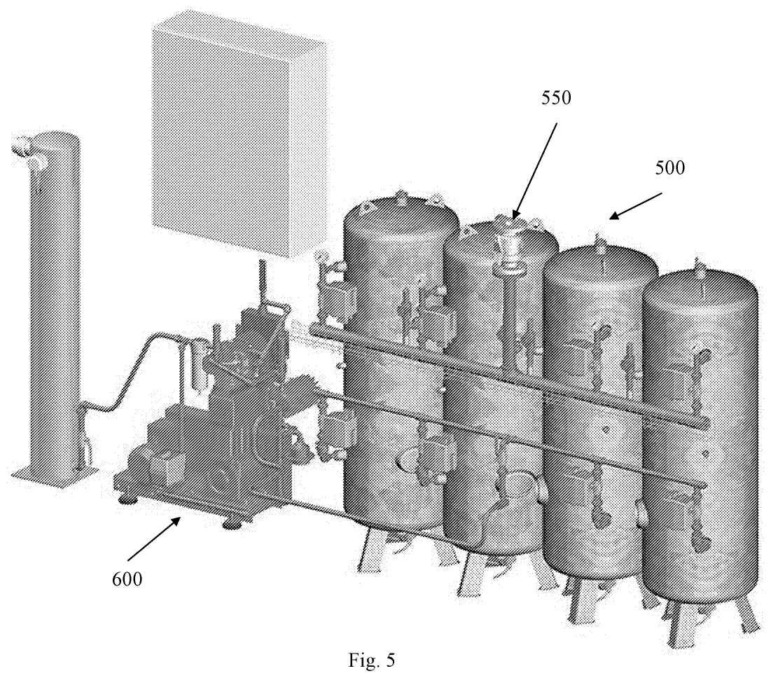

As shown in FIG. 4, after the cyclotron operation creates the particular isotope desired, the resultant gaseous waste is analyzed to detect if any residual radiation is present, with the portion of the exhaust gas which is fee of radiation being vented into a HVAC system, and the portion of the exhaust gas which contains radiation being segregated or filtered for further treatment, as described further below.

According to the present disclosure, the cyclotron system includes a gas waste disposal unit that includes a storage tank(s) 500 for storing the gaseous waste having an outlet valve 550 for removing/venting the gaseous waste from the storage tank. The storage tank(s) includes an inlet valve(s) for controlled filling of the gaseous waste. A compressor 600 is connected to the inlet valve for pressurizing the gaseous waste in the storage tank, and a sensor/controller can be included for controlling the compressor and for determining the pressure of the gaseous waste, such that the compressor can be intermittently activated for maintaining the pressure of the gaseous waste in the storage tank.

The ambient atmosphere is accessible through the outlet valve when the outlet valve is open, with the pressure differential (i.e. elevated pressure within the storage tank) driving the gaseous waste from the storage tank by escaping to the ambient atmosphere through the outlet valve. In an exemplary embodiment, the outlet valve can be permanently positioned in at least partially open position (e.g. half-way open position) which allows a continuous stream of exhaust air to exit the storage tank, thereby reducing/eliminating any sudden rush of air which may cause an imbalance in the ambient air conditions. In some embodiments, the valve can be a binary valve having only open and closed configurations; in some embodiments a servo valve can be provided with adjustable orifice sizes. For purpose of illustration and not limitation, in an exemplary embodiment the storage tank can contain gas at approximately 2-18 bars (e.g. 15 bars). In an exemplary embodiment, two storage tanks are employed, each providing a gas capture/trapping dwell time of approximately 45 minutes in order to capture the volume of radioactive gas that may be present in the hot cell of the cyclotron.

The system disclosed herein can be employed for treatment and release of exhaust gases from a variety of radioisotopes. For purpose of illustration and not limitation, some exemplary radioisotopes include, for example, ¹⁵O, ¹¹C gas, liquid ¹⁸F, Solid TRG, ⁶⁸Ga, ⁶⁷Ga, ⁸⁹Zn, ⁶⁴Cu, ¹³N, ¹²⁴I, ¹⁷⁷Y, etc.

In accordance with an aspect of the present disclosure, a method for collecting, storing and releasing of radio-isotopic gaseous waste material is provided which includes providing a storage tank with an inlet valve and an outlet valve to regulate filling rate of the gaseous waste within the storage tank. Additionally, a compressor can be connected to the inlet valve of the storage tank for controlling the pressure of the gaseous waste. As the gaseous waste is delivered to the storage tank through the inlet valve the compressor operates to increase the pressure of the gaseous waste. In some embodiments, the compressor operation can be delayed until the filling operation is completed. The compression cycle(s) can be monitored with sensors that maintain the internal tank pressure within a predetermined range. The gaseous waste can be stored within the tank for a predetermined time period (e.g. depending on the particular radioisotope half-life).

Venting or exhaust of the gaseous waste is permitted via operation of the outlet valve 550. Although the exemplary embodiment shown depicts only a single outlet valve, additional valves can be included, if desired. Upon exiting the valve(s) the exhaust gas travels through the sound suppression device 700 (e.g. muffler) connected, or coupled to permit gas transfer, with the outlet valve 550. The venting operation (and its termination) can be performed manually through predetermined set points, on a scheduled basis, or automatically, e.g., if the pressure within the storage tank reaches approximately 15 bars, it automatically switches over to the second tank. Irrespective of the particular venting operation employed, the stored gas is only permitted to be released to the ambient when the level of radioactive decay, which is based on a pre-programmed timer (commencing as soon as the minimal internal pressure is achieved within the storage tanks 500), reaches an approved level.

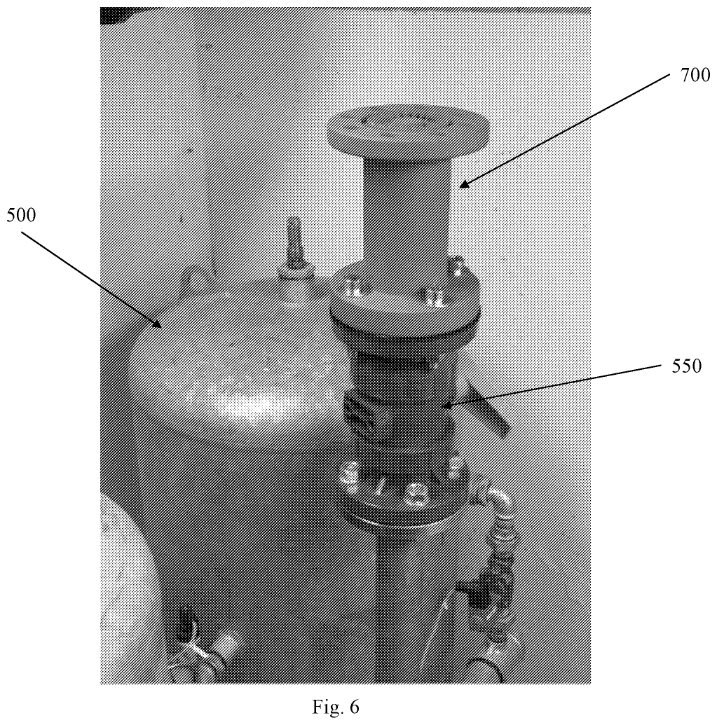

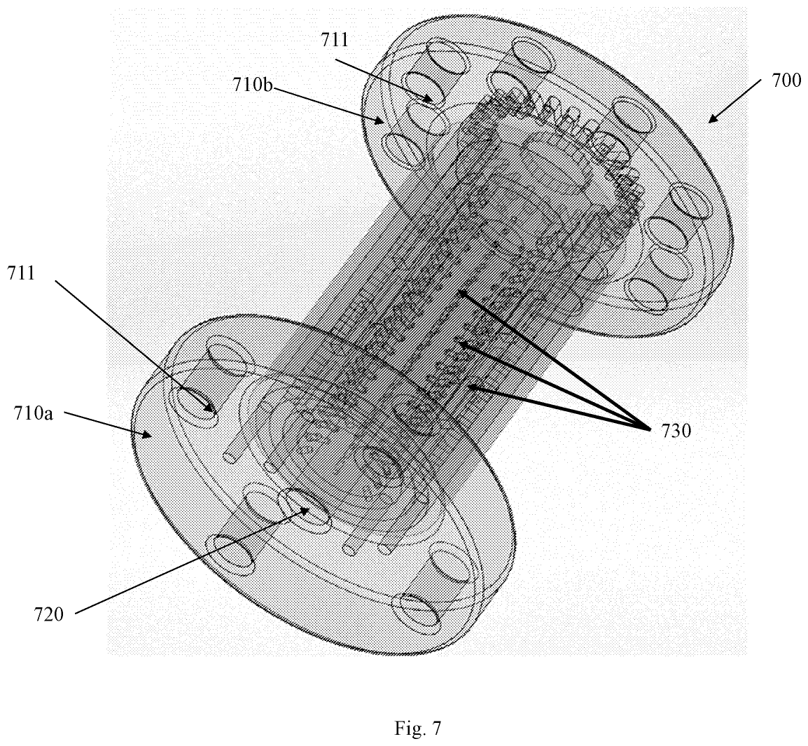

In an exemplary embodiment, the sound suppression device 700 is disposed above the outlet valve 550. As shown in FIG. 7, the suppression device 700 can be formed in a cylindrical shape having a bottom flange 710a and top flange 710b. The flanges 710a,b can be formed with equivalent dimensions (e.g. circumference, thickness) and include a plurality of apertures 711 which extend through the thickness of the flange. The apertures on the lower flange 710a can be sized and positioned to receive the hardware (e.g. bolts, screws, etc.) for fastening to the outlet valve 550, as shown in FIG. 6. The upper flange 710b can include a greater number of apertures than the lower flange 701a, this allows for the upper flange 701b to be connected to an exhaust pipe, if desired. In some embodiments the upper and lower flanges 710a,b, can be formed with identical features such that the sound suppression device can be easily mounted to the outlet valve 550 on either end.

The sound suppression device 700 has a primary internal channel 720 having a diameter equivalent to the diameter of the exhaust valve 550. In the exemplary embodiment, this primary internal channel 720 is configured as a linear channel aligned with the longitudinal axis of the device 100. Additionally, the sound suppression device can include a plurality of diffusion channels 730, 740, 760 which are distributed along the length of the device, and connected with the primary internal channel 720 to permit gas passage therebetween. The diffusion channels can be linear or non-linear (e.g. curved, helical, etc.) provided that the sum of diffusional channels is greater than the diameter of the primary internal channel 720. In the exemplary embodiment shown, a first series of diffusion channels 730 extend radially from the primary channel 720, though alternative configurations can be employed. Similarly, in the exemplary embodiment shown the diffusion channels 730 are uniformly distributed along the longitudinal axis of the device, however the density of these channels can be varied (e.g. randomly, or in a gradient fashion) to create zones of more/less diffusion channels along the length of the device.

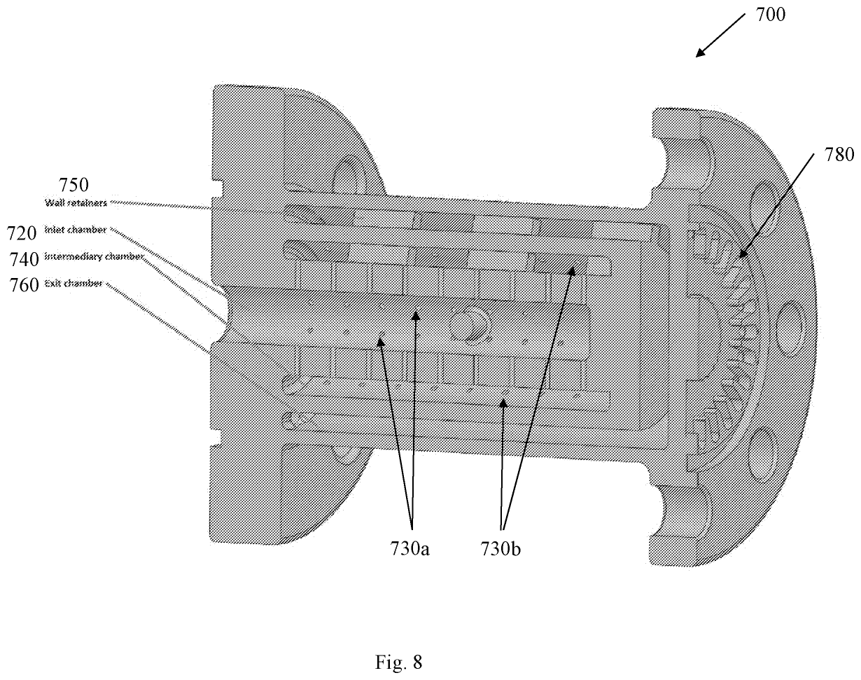

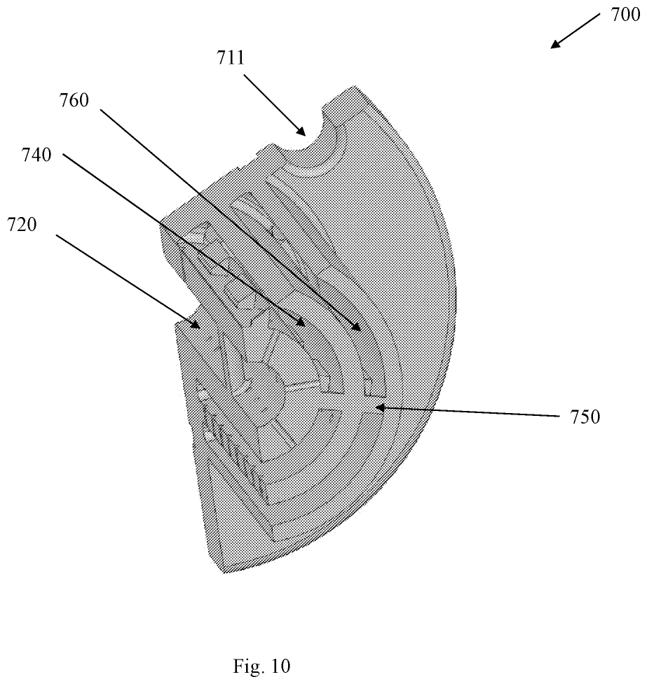

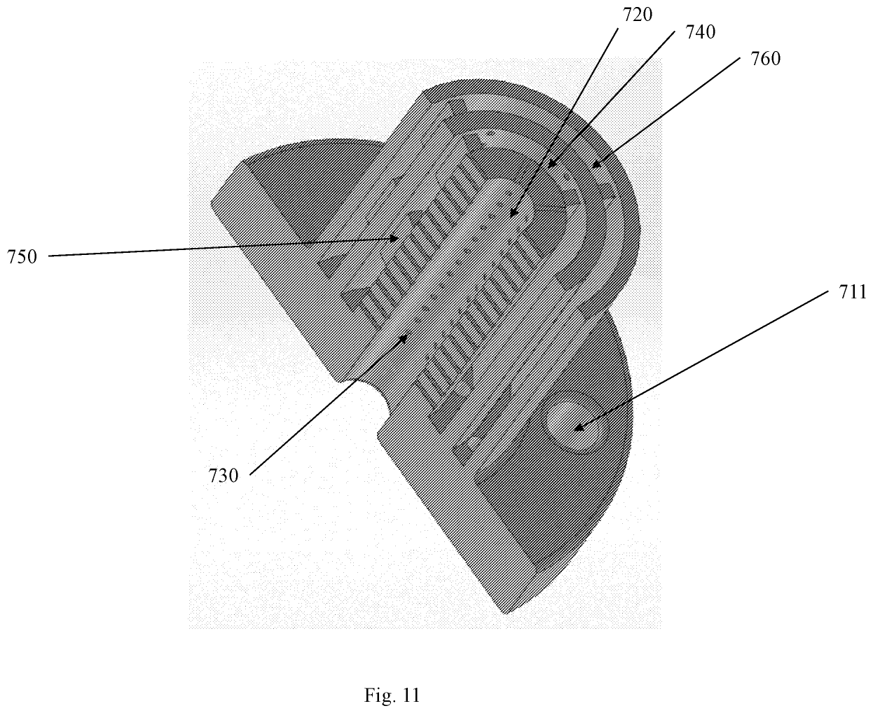

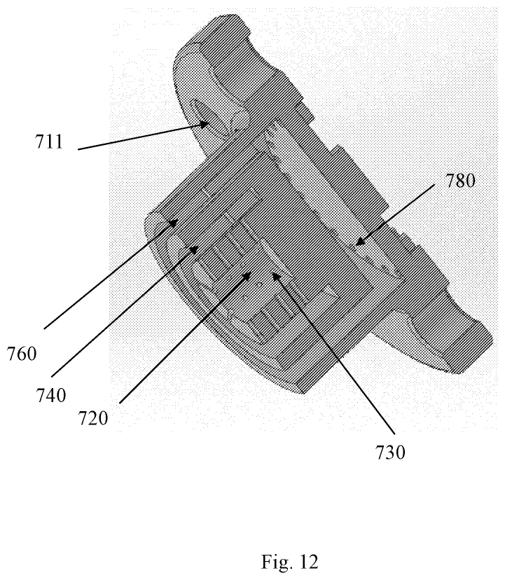

As shown in the exemplary embodiment of FIGS. 8-12, the diffusion channels are connected to the primary channel 720, with an intermediary chamber (or “channel” which will be used interchangeably herein) 740 disposed radially outward of primary chamber/channel 720, and an exit chamber/channel 760 disposed radially outward of intermediary chamber 740. In this exemplary embodiment, the sound suppression device is configured such that the channels 720, 740 and 760 are concentrically arranged with radially extending interconnecting channels 730 permitting gas to pass therebetween. In some embodiments the channels can be staggered or longitudinally offset such that channel 720 extends to an end of the flange, while channels 740 and 760 extend longitudinally beyond channel 720, as shown. A plurality of wall retainers 750 are provided to maintain the space between channels 720, 740 and 760. In the embodiment shown channels 740 and 760 are of equivalent volume/diameter, though alternative configurations can be employed if desired.

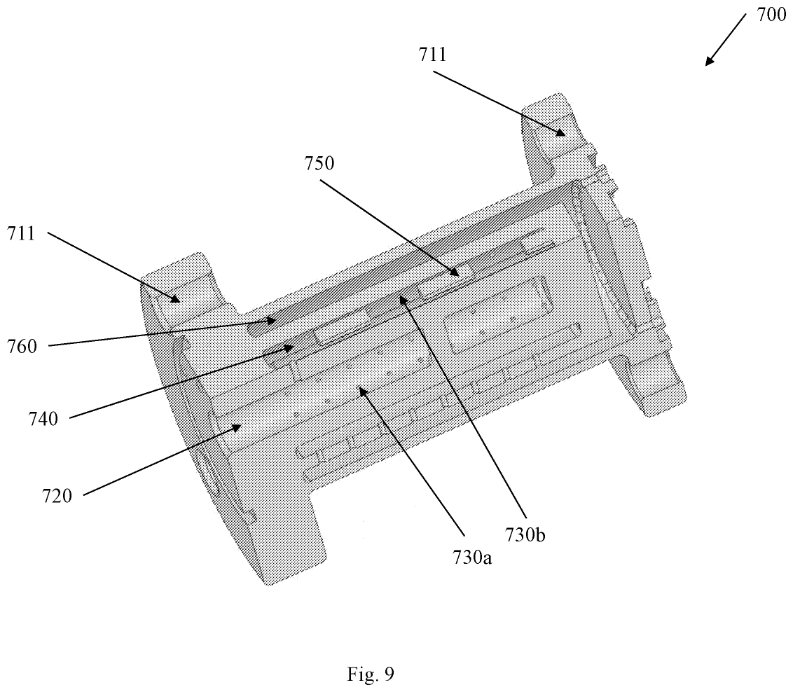

During operation, gas travels from the exhaust valve through into primary channel 720, through diffusion channels 730a into intermediary chamber/channel 740, and thereafter through diffusion channels 730b into exit chamber/channel 760, as shown in FIGS. 8-9. From the exit channel 760 the gas escapes to the ambient atmosphere through fins 780. This tortuous path serves to reduce the amount of energy (e.g. pressure) in the exhaust gas so that, upon exiting the sound suppression device through fins 780, the pressure differential is reduced thereby muffling the sound generated by the exhaust gas.

The sound suppression device 700 can be formed of a variety of methods and materials, for purpose of illustration and not limitation, an exemplary embodiment of the sound suppression device 300 can be formed via additive manufacturing (i.e. 3D printing) of nylon.

In operation, when the high pressure exhaust gas is vented through the exhaust valve, and subsequently the sound suppression device 700, the high pressure and high speed exhaust gases travel through the primary and secondary channels 730 of the sound suppression device, which effectively increase the distance the exhaust gas must travel before exiting into the ambient atmosphere. This reduces the noise generated by the exhaust gas from conditions of greater than 125 decibels (without the sound suppression device disclosed herein) to approximately 65-75 decibels (with the sound suppression device disclosed herein attached to the outlet valve). This significantly reduces the risk of hearing damage to personnel operating the cyclotron, or even standing several meters away from the cyclotron.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A cyclotron system comprising:
a cyclotron unit for preparing a radioisotope;
at least one storage tank for storing a radioactive gas resulting from preparation of the radioisotope;
a compressor connected with the storage tank(s);
an exhaust valve connected with the storage tank(s); and
a sound suppression device connected with the exhaust valve.

2. The system of claim 1, wherein the compressor compresses the internal pressure of the storage tank above approximately 10 bars.

3. The system of claim 1, wherein the sound suppression device has a primary internal channel with a diameter approximately equivalent to the diameter of the exhaust valve.

4. The system of claim 3, wherein the primary internal channel is configured as a linear channel aligned with the longitudinal axis of the sound suppression device.

5. The system of claim 3, wherein the sound suppression device includes a plurality of diffusion channels distributed along the length of the device and connected with the primary internal channel.

6. The system of claim 5, wherein the diffusion channels extend radially from the primary channel.

7. The system of claim 5, wherein diffusion channels are uniformly distributed along the longitudinal axis of the sound suppression device.

8. The system of claim 3, wherein exhaust gas is vented through the sound suppression device traveling through the primary and secondary channels.

9. The system of claim 1, wherein the sound suppression device generates an exhaust gas of approximately 65-75 decibels.

10. The system of claim 1, wherein the sound suppression device is made of nylon.

11. A sound suppression device, the sound suppression device configured for coupling to an exhaust valve of a cyclotron comprising:
a first flange, the first flange having a plurality of apertures disposed therein;
a second flange, the second flange having a plurality of apertures disposed therein;
a generally cylindrical length defined between the first and second flanges;
wherein a primary internal channel extends throughout the cylindrical length of the device.

12. The device of claim 11, wherein the diameter of the primary internal channel is approximately equivalent to the diameter of an exhaust valve of the cyclotron.

13. The device of claim 11, wherein the primary internal channel is configured as a linear channel aligned with a longitudinal axis of the device.

14. The device of claim 11, wherein the device includes a plurality of diffusion channels distributed along the length of the device, each connected with the primary internal channel.

15. The device of claim 14, wherein the diffusion channels extend radially from the primary channel.

16. The device of claim 14, wherein diffusion channels are uniformly distributed along the longitudinal axis of the device.

17. The device of claim 14, wherein diffusion channels have uniform diameters.

18. The device of claim 14, wherein diffusion channels include a first set of channels having a first diameter, and a second set of channels having a second diameter.

19. The device of claim 14, wherein diffusion channels are linear channels.

20. The device of claim 11, wherein the second flange has a greater number of apertures than the first flange.
